(19) United States
(12) Patent Application Publication
(10) Pub. No.: US 2022/0410051 A1
(43) Pub. Date: Dec. 29, 2022

(54) Extraction hood

(71) Applicant: Pal Costruzioni Srl, Bovolenta (PD) (IT)

(72) Inventor: Paolo Donola, Bovolenta (PD) (IT)

(21) Appl. No.: 17/756,635

(22) PCT Filed: Oct. 1, 2020
(86) PCT No.: PCT/IB2020/059209

§ 371 Date: May 28, 2022

(30) Foreign Application Priority Data
Dec. 20, 2019 (IT) 102019000024928

(51) Int. Cl. B01D 46/00; F24C 15/20; B01D 39/20; B01D 46/121; B01D 46/48; A61L 9/20

(52) U.S. Cl. CPC B01D 46/0028; F24C 15/2035; B01D 39/2027; B01D 46/121; B01D 46/0038; B01D 46/48; A61L 9/20; B01D 46/58

(57) ABSTRACT

An extraction hood includes a casing having one or more suction openings or mouths and a connection system to an outlet duct for the removal of the drawn air filter. Inside the casing there are one or more filters and one or more UV lamps designed to be used for disinfection, for the abatement of smells and greases, and for self-cleaning purposes.

This patent relates to extraction hoods and in particular concerns a new extraction or suction hood, mainly for professional use, equipped with a self-cleaning system for disinfection and the abatement of odors, vapors, fats, and grease.

Extraction hoods used to extract fumes, vapors and gases from an environment, and to discharge them into the atmosphere are well known in the prior art.

Extraction hoods for industrial use installed in production plants where the production involves the creation of fumes, vapors and gases that can be harmful to personnel are also known to the prior art.

These fumes are discharged directly into the atmosphere, or more often they are sent to a purification and abatement system before the purified air flow is discharged into the atmosphere.

Extraction hoods for professional kitchens are also known in the prior art, normally installed above cooktops, ovens, fryers, and other devices for cooking food.

Extraction hoods of this type must not only draw in the fumes produced during cooking, but must also capture the grease, humidity and particles present in the cooking fumes.

Normally, professional extraction hoods comprise a casing made of metal material, for example with a substantially box-like shape to be installed against the ceiling, or of a generally trapezoidal shape to be installed on a wall.

On the lower wall of the casing there are one or more air intake openings. The casing houses one or more filters and deflectors to convey the flow of air towards said filters. Finally, the casing is connected to an exhaust duct through which the extracted and filtered air flow is sent for subsequent treatments or to be discharged into the atmosphere.

The suction system, which generally comprises at least one suction fan and a power supply system for the fan itself, is installed downstream of said filters.

Extraction hoods in the prior art also include the function of drawing clean air into the environment in which they are installed, and where the quantity of clean air introduced into the aforementioned environment is generally lower than that extracted, to ensure a slight negative pressure in the kitchen.

Professional extraction hoods of the prior art include the so-called “labyrinth” or baffle filters, which comprise a grid made up of steel pathways, shaped and joined to each other making the air flow follow an circuitous route. The particles transported by the air flow collide against the filter surfaces and, sliding along the channels, are collected in special drainage channels located at the bottom of the filters.

In this way, the grease, water, and heavier particles carried by the air flow are captured and collected.

Alternatively or in addition to these baffle filters, the use of mesh filters is also known in the prior art. They comprise a plurality of superimposed steel mesh having the task of capturing the larger particles in the air flow, and are also intended as a barrier to flames and humidity.

Thus particles of grease, water and liquid fluids in general accumulate on their surface, where microorganisms such as bacteria and germs inevitably develop and proliferate, also thanks to the favorable conditions resulting from the heat and humidity found inside such systems.

To avoid this proliferation, as well as to ensure the efficiency of the filtration systems, it would be necessary to periodically clean all the parts that make up the hood, particularly upstream of the filters, and in particularly the filters themselves, where most of the particles are collected.

Filter cleaning operations are very complicated since these filters ought to be removed and completely degreased, including the areas that are difficult to reach. Due to the size of these filters, it is not always possible to wash them in a dishwasher.

In fact, most of the time the filters are replaced with new ones, but in reality this replacement operation is performed less often than is actually necessary.

In addition, the fumes treated are usually quite odorous. The filters normally used are able to retain most of the larger grease and vapor particles, while the fraction containing the finer particles, in the form of aerosols, passes through the filters and is discharged into the atmosphere, taking those odors with it. These fine particles adhere to the internal surfaces of the hood and inside the exhaust ducts or even near the outlet of the exhaust itself.

The accumulation of these fine particles over time leads to bacterial proliferation, the decomposition of the organic substances, and the onset of even nauseating odors. To obviate the aforementioned drawbacks, an extraction hood with a disinfection system and odor, vapor and grease abatement system was developed and constructed.

The main object of the new extraction hood is to carry out self-cleaning and odor abatement functions, since it uses the combined action of filters and UV emissions to degrade almost all fats, vapors, and odors.

Another important object of the new hood is to limit the proliferation of microorganisms in the hood assembly and more specifically in the filters.

Yet another object of the present invention is to further limit the proliferation of microorganisms even downstream of the filters, thanks to the particular combination of types of filters used to maximize the filtration efficiency.

One advantage of the present invention is that of facilitating and speeding up all cleaning operations of the hood since the innovative and synergistic combination of filtration and irradiation with UV rays degrades the fats and grease carried by the extracted fumes, which are pulverized and easy to remove.

These and other direct and complementary objects are achieved by the new extraction hood equipped with a disinfection system which in its main parts comprises:
a substantially box-shaped casing, on which there are one or more intake openings or inlets and means for connecting at least one exhaust duct for removing the flow of extracted air;
one or more filters installed inside said casing;
walls or deflectors installed inside said casing to guide the flow of extracted air toward and through said at least one filter;

and where one or more lamps or means suitable for emitting UV rays are also installed inside said casing.

Inside said casing there is also at least one channel or collection tank for the filtered material that drips from said filters.

Said one or more UV lamps are suitably oriented so as to irradiate at least one surface of at least one of said filters and/or the interior of said at least one channel or collection tank.

More preferably, said one or more UV lamps are arranged so as to irradiate all the surfaces inside said casing and in particular the upper surfaces of said filters, and the interior of the channel.

Combining the filtering action with the disinfectant action carried out by the UV lamps, offers adequate treatment to reduce grease, vapors and odors of the extracted fumes: the larger fraction contained in the fumes is in fact retained by said filters, while the finer particles of vapors and fat aerosols are degraded by the action of the UV rays. The residue of the degradation, minimized and odorless, is collected in part in said at least one channel and in part discharged into the atmosphere through exhaust ducts.

Therefore, the hood has a self-cleaning function, since the internal walls remain clean because the particles of fat and grease are degraded and do not adhere to them. The discharged air flow will also be free of odors, as well as of grease and vapor particles which would otherwise adhere to the walls of the exhaust ducts, producing the known undesirable effects.

The proliferation of microorganisms is also limited, so that the necessary hygiene conditions are guaranteed and preserved for longer than traditional filtration systems.

In the preferred embodiment, the new extraction hood comprises one or more labyrinth or baffle filters and/or one or more filters of the mesh type. Said filters are preferably of the type suited to be irradiated with UV rays, that is, certified for this purpose.

Said filters are preferably arranged with a certain inclination inside the casing, so that the material captured by the filters slides downwards in the direction of inclination, and is collected in said at least one channel or collection tank.

In the preferred embodiment, said filters are arranged in two parallel rows inclined so as to form a sort of V-shaped abatement chamber, where said channel or collection tank is installed between the two rows of filters.

Said UV lamps are installed inside said abatement chamber so as to preferably irradiate all the surfaces of the chamber itself.

The characteristics of the new extraction hood will be better explained by the following description with reference to the drawing, attached by way of a non-limiting example.

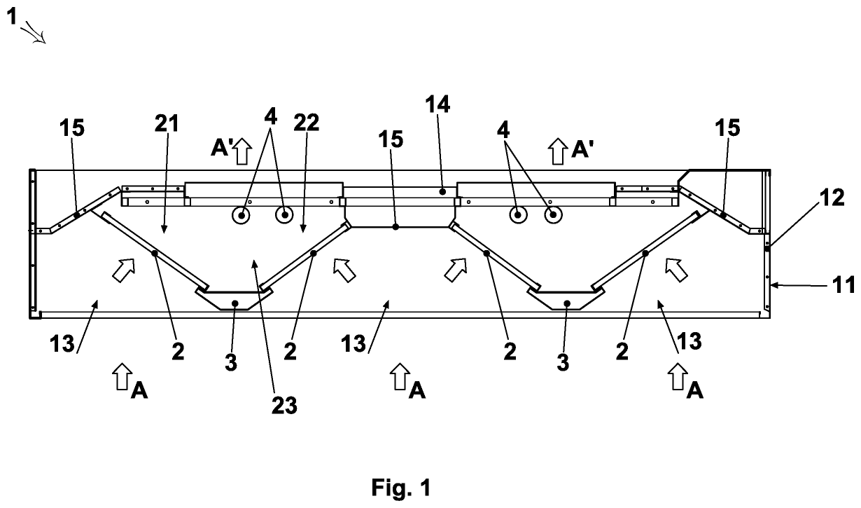

FIG. 1 shows one configuration of the new extraction hood (1) in a non-limiting example.

The new extraction hood (1) comprises a substantially box-shaped casing (11), determining an internal volume (10) in which the filtration of an air flow (A) takes place, for example the fumes generated by a cooktop.

Said casing (11) comprises one or more openings (13) or intake openings at its bottom, through which the air flow (A) to be filtered enters.

Conveniently, said air flow (A) is generated by an aspiration system not shown in the FIGURES.

Said casing (11) is laterally delimited by walls (12) and at the top by a cover or ceiling (14), wherein said walls (12) and/or said ceiling (14) are equipped with means for constraining the casing (11) to the ceiling or a wall and to connect at least one channel for the discharge of the filtered air flow in question.

Inside said casing (11) there are one or more filters (2) for filtering the air and walls or deflectors (15) installed inside said casing (11) to convey the flow of extracted air (A) through said filters (2).

Said filters (2) are preferably of the labyrinth or baffle type and/or of the mesh type, preferably made of stainless steel and structured and certified to be irradiated by UV rays.

Preferably, said filters (2) are constrained inside said casing (11) by removable means, in order to enable them to be removed and replaced. For example, said filters (2) are secured by guides.

In the example shown in FIG. 1, said filters (2) are arranged inclined inside said casing (11), for example on one or more pairs of parallel rows (21, 22) and inclined so as to create a substantially V-shaped abatement chamber (23) between each pair of rows (21, 22).

At the lowest point of said abatement chamber (23), between the two rows (21, 22) of filters, at least one channel or collection tank (3) is mounted for the material carried by the air flow (A) and captured by the filters (2), wherein said material normally comprises particles of fat and water, particularly in the case of fumes generated by a cooktop or in a kitchen in general.

Said channel or collection tank (3) can also be removed to facilitate its cleaning. Inside said casing (11) there are also one or more UV lamps (4) oriented so as to preferably irradiate at least one part or surface of at least one of said filters (2) and preferably all the internal surfaces of said abatement chamber (23).

In the preferred embodiment shown in the FIGURE, said UV lamps are installed above said abatement chamber (23) located between the two rows (21, 22) of said filters (2), so that the light emitted by the lamps substantially irradiates the entire surface of the filters (2), at least on the filtered air flow (A′) outlet side.

Said UV lamps (4) are in particular arranged so that the light emitted also irradiates the inside of said at least one channel or collection tank (3) of the filtered material. In particular, at least one of said UV lamps (4) is, for example, installed above said channel or collection tank (3) for the filtered material.

Therefore, with reference to the preceding description and the attached drawing the following claims are made.

The invention claimed is:

1. An extraction hood (1) comprising:
a substantially box-shaped casing (11) having one or more suction openings or mouths (13) and connection means to at least one outlet duct designed to remove a drawn air flow;
one or more filters (2) positioned inside said casing (11), wherein at least one of said one or more filters (2) is arranged inclined inside said casing (11), so that material held by said one or more filters (2) slides downwards in a direction of inclination and is collected in at least one collection channel or container (3) arranged in a corresponding position inside said casing (11); and
one or more UV lamps installed inside said casing (11).

2. The extraction hood (1) according to claim 1, wherein said one or more filters are completely or partially made of stainless steel.

3. The extraction hood (1) according to claim 1, wherein at least one of said one or more filters (2) is a baffle filter.

4. The extraction hood (1) according to claim 1, wherein at least one of said one or more filters (2) is a mesh baffle filter.

5. The extraction hood (1) according to claim 1, wherein at least one of said one or more UV lamps (4) is oriented to irradiate an inside of said casing (23), at least part of a surface of at least one of said one or more filters (2), or at least an inside of said at least one collection channel or container (3).

6. The extraction hood (1) according to claim 1, wherein at least one of said one or more UV lamps (4) is placed above said at least one collection channel or container (3) designed to collect the filtered material.

7. The extraction hood (1) according to claim 1, wherein said one or more filters (2) are configured or arranged on one or more pairs of parallel rows (21, 22) and inclined so as to form a substantially V-shaped abatement chamber (23), and wherein said at least one collection channel or container (3) is positioned between each pair of rows (21, 22) of said one or more filters (2).

8. The extraction hood (1) according to claim 7, wherein said one or more UV lamps are distributed in said at least one abatement chamber (23) in such a way as to irradiate all inner surfaces of said abatement chamber (23).

9. The extraction hood (1) according to claim 1, wherein at least one of said one or more filters (2) or said collection channel or container (3) are constrained inside said casing (11) through reversible constraining means, so as to allow said one or more filters (2) to be extracted and, if necessary, replaced.

10. The extraction hood (1) according to claim 1, wherein said casing (11) is delimited laterally by walls (12) and has a top cover or ceiling (14), and wherein said walls (12) and/or said ceiling (14) are provided with means designed to constrain the casing (11) to the top cover or ceiling or to the walls and to connect the at least one outlet duct to enable a movement of the filtered air flow.
